(19) United States
(12) Patent Application Publication
(10) Pub. No.: US 2023/0371819 A1
(43) Pub. Date: Nov. 23, 2023

(54) EARPLUG COMPRISING A HEAT DETECTOR AND A PULSE MONITOR AND A METHOD OF MONITORING A SUBJECT OF HIS BODY HEAT

(71) Applicant: WELL BEING DIGITAL LIMITED, New Territories (HK)

(72) Inventor: Ming Yip Wallace WONG, Kowloon (HK)

(21) Appl. No.: 18/248,327

(22) PCT Filed: Aug. 3, 2021
(86) PCT No.: PCT/CN2021/110263

§ 371 Date: Apr. 7, 2023

(30) Foreign Application Priority Data
Oct. 7, 2020 (HK) 32020017309.6

(51) Int. Cl. A61B 5/01; A61B 5/024; A61B 5/0245; A61B 5/1455; A61B 5/00; A61B 5/026

(52) U.S. Cl. CPC A61B 5/01; A61B 5/02416; A61B 5/02438; A61B 5/02455; A61B 5/14551; A61B 5/6817; A61B 5/7275; A61B 5/746; A61B 5/0261

(57) ABSTRACT

A device 2300 to be inserted into the ear canal having at least two thermistors 205, 207 for observing a temperature gradient in the ear canal of a user, and a photoplethysmography sensor 215, 217. Changes in the temperature gradient, heart rate and magnitude of the pulse are monitored for determining the risk of the user in facing an imminent heat stroke.

### FIELD OF INVENTION

This invention relates to the field of continuous monitoring of bodily temperature. In particular, the invention relates to apparatuses and methods for monitoring danger caused by or accompanied by changes in bodily temperature, such as heatstroke.

### BACKGROUND OF THE INVENTION

Conventionally, body temperature is measured using a mercury thermometer. The mercury thermometer has a glass bulb filled with mercury which overflows from the bulb into a capillary tube. The mercury expands and contracts in the capillary tube according to heat transmission into or away from the mercury across the bulb wall. The bulb is placed against the body of a subject whose temperature is to be measured, and is typically inserted into a crevice of the body, such as beneath the tongue, in an arm pit, or into the rectum. The choice of location depends on the age of the subject. Time is required for heat to transfer from the body into the mercury and to reach equilibrium so that expansion of the mercury stabilises. Hence, it is often time consuming and uncomfortable for the subject who is inserted with a mercury thermometer to wait while his temperature is being read.

The mercury thermometer has a serous disadvantage. It cannot be used to monitor the subject's temperature over a period of time continuously. The mercury thermometer is only useful for providing a single reading of temperature, of a discrete moment.

The tympanic infrared thermometer has been proposed, which measures body temperature more comfortably by detecting infrared emissions from the ear drum. The ear drum is also known as the tympanic membrane. The tympanic infrared thermometer is commonly seen as a handheld device in clinics, and has a spout containing an optical detector. The spout is shaped to be inserted into the earhole. The optical detector detects infrared emissions from the tympanic membrane, and the tympanic infrared thermometer very quickly deduces body temperature from the emissions based on calibration. An advantage of the tympanic infrared thermometer is that body temperature is read very quickly, virtually in less than a second. This relieves the subject of the need to wait while his temperature is being read, unlike using the mercury thermometer. However, it is difficult to secure a line of sight from the optical detector at the opening of the ear hole to the tympanic membrane, especially if the handheld device is not handled with experience. Furthermore, such a handheld device is not designed to be worn by a subject, and is therefore not useable for continuous body temperature monitoring. Like the mercury thermometer, the handheld tympanic infrared thermometer is only useable to obtain a single temperature reading.

It has been a desire for subjects who are firemen to have their temperature monitored during their training or work, in order to assess their risk of serious heat injury. In a hot environment with intense work stress, a fireman is unlikely to notice that he is running a fever and that he is in danger of heat injury. It is also difficult for his supervisor, who is usually nearby conducting the fire rescue carried out by the fireman but distanced from the fire itself, to monitor the fireman's condition by relying on observation skills of other firemen in the team. If a fireman collapses from heat injury, his team mates will have to turn to focus on rescuing him instead of fighting fire.

It has been proposed to configure the tympanic infrared thermometer into an ear-wearable design. A fireman can then wear it in one of his ears during fire rescue, so that his temperature may be monitored continuously throughout the rescue. However, the ear-wearable design suffers the same inherent difficulty of securing a line of sight between the optical detector and the tympanic membrane. Moreover, movements of the fireman can easily break the line of sight.

Any device that monitors body temperature accurately and precisely is a ‘sensitive’ thermometer, and has to be calibrated. However, instrument calibration is subject to drifting. This imposes a need for regular re-calibration to maintain accuracy. If a sensitive device is used in a busy situation that subjects the device to the forces of a lot of movements, sudden and significant calibration drifts may occur. If such a sensitive thermometer is relied upon to raise an alarm when the subject's temperature is too high, a calibration drift could cause false alarm or cause a valid alarm not to be raised. Hence, overly sensitive thermometers are not suitable for use in continuous monitoring of firemen's temperature during fire rescues.

Ear-based temperature monitoring devices have been proposed which monitors the temperature in the ear canal of the subject. The devices are robust, rugged and suitable for deployment on subjects such as firemen in a harsh condition. However, it is desirable that these devices are improved to be even more accurate.

### STATEMENT OF INVENTION

In the first aspect, the invention proposes an earplug comprising a heat detector; a pulse monitor; wherein the heat detector is capable of obtaining an indication of the level of heat in the air of the ear canal of a subject wearing the earplug at the same time as the pulse monitor is obtaining an indication of the heart rate of the subject. The invention thereby provides a possibility of a non-invasive, convenient and wearable device that is able to monitor both the pulse and the temperature of a subject. As there are people whose temperature may be significantly elevated due to their activities or the environment they are in, but who are not in any immediate danger because their pulse is regular and not overly fast. Alternatively, there maybe people whose temperature has increased just very slightly but because of an accompanying increase in heart rate, these people should be checked by medical personnel to ensure that they are not in danger. The invention provides the advantage of allowing such people to be identified by a single wearable instrument.

The earplug can be part of an earphone, part of a hearing aid or just a plug for the ear canal. The ear canal is a good place for monitoring a person physiologically without getting in the way of daily life. Wrist worn devices, by way of example, may be wet when hand washing at the lavatory. Furthermore, it is easier to secure devices to the ear than tying devices to the wrist by a belt.

Typically, the heat detector is formed of at least two heat sensors arranged to be spaced apart by a pre-determined distance such that the at least two heat sensors are capable of measuring the temperature of the air in at least two respective locations in the ear canal; wherein the temperature of the air in at least two respective locations being suitable for deducing the temperature gradient in the ear canal to provide the indication of the level of heat in the ear canal. Preferably, the at least two heat sensors are semiconductor temperature sensors that can fit into the part of the earplug that is intended to be placed into the ear canal. Using a temperature or heat gradient of the air in the ear canal as an indication of the body heat or the temperature of the subject is more robust than most direct temperature measurement used in the prior art.

Preferably, the pulse monitor is a photoplethysmography sensor or a ballistocardiography sensor. Alternatively, other methods of detecting the pulse can be used, such an electrocardiography sensor. In particular, the photoplethysmography sensor is a very rugged sensor and also allows the earplug to be worn by a very active subject.

Optionally, the earplug further comprises an extension suitable for placement into the ear canal; the extension having a first side and a second side; the first side and the second side having dimensions such that the first side is closer to the ear canal wall and the second side is further from the canal wall; the pulse monitor being placed on the first side; and the heat detector being placed on the second side. Typically, the second side is in a depression on the surface of the extension. This provides the advantage of an increased chance that the heat detector is able to measure the temperature of the air in the ear canal instead the temperature of the ear canal wall or tissue. Alternatively, the earplug further comprises an extension for placement into the ear canal, the heat detector being on the extension; earplug having a plug portion for remaining outside the ear canal when the earplug is worn by the subject, the pulse monitor being on the plug portion; the plug portion having dimensions suitable for urging the pulse monitor into contact with the tragus of the ear when the extension is placed into the ear canal; wherein the indication of the heart rate of the subject is obtained from the tragus of the ear of the subject.

Optionally, the earplug also comprises a flexible printed circuit board; the heat detector and the pulse monitor being components on the flexible printed circuit board; the flexible printed circuit board folded to define a space for accommodating the heat detector, and the flexible printed circuit board folded to fit inside the earplug; such that when the earplug is worn by the subject, the heat detector is placed into the ear canal.

Preferably, the flexible printed circuit board has rigidized portions. Typically, the flexible printed circuit board is reinforced by one or more layers of a hard substrate such that a respective one or more portions of the flexible printed circuit board are more rigid than the other portions of the flexible printed circuit board; wherein the flexible printed circuit board is folded such that the rigid portions of printed circuit board cooperate to define the space for accommodating the heat detector.

In prior art use of a flexible printed circuit board, there is no motivation or need to have any rigid parts. Hence, rigidizing portion of the flexible printed circuit board allows one to fold the flexible printed circuit board into a structure that provides an somewhat semi-enclosed space, or a shall, and which does not collapse and become flattened. This novel approach teaches against the tendency to make the flexible printed circuit board as flexible as possible and allows sensitive electronic components to be wrapped around by the rigidized portions and be protected.

In a second aspect, the invention proposes a method for monitoring a subject of his body heat, comprising the steps of: obtaining an indication of the level of heat in the ear canal of the subject at the same time as obtaining the heart rate of the subject; and raising an alert if the heart rate is higher than a pre-determined upper threshold heart rate for the level of heat in the ear canal; or raising an alert if the heart rate is lower than a pre-determined lower threshold heart rate for the level of heat in the ear canal. This method does not amount to any diagnosis as no diagnostic conclusion is made. However, this method allows the subject's condition to be brought to attention so that medical personnel may check on the subject.

Typically, the indication of the level of heat is the temperature gradient in the canal of the ear. Preferably, the step of obtaining an indication of the level of heat in the ear canal further comprises detecting a change in the temperature gradient in the canal of the ear.

Preferably, the method further comprises the steps of: observing the indication of the level of heat at the same time as observing the amount of subcutaneous blood in the subject; and raising an alert if the heart rate is higher than a pre-determined upper threshold heart rate for the level of heat in the ear canal without an accompanying increase in the amount of subcutaneous blood in the subject to a pre-determined threshold amount. This feature provides that the effectiveness of heat dissipation at the subject's skin may be estimated by the subcutaneous blood content or subcutaneous blood flow.

Preferably, the step of observing the amount of subcutaneous blood of the subject over the same period of time comprises: observing the amount of absorption of a light transmitted into the skin of the subject. More preferably, the step of observing the amount of subcutaneous blood of the subject comprises: observing the ratio of deoxygenated haemoglobin to oxygenated haemoglobin; taking an increase in the ratio of deoxygenated haemoglobin to oxygenated haemoglobin to mean an increase in subcutaneous blood; and taking a decrease in the ratio of deoxygenated haemoglobin to oxygenated haemoglobin to mean a decrease in subcutaneous blood. As the skilled man would understand, blood on the surface of the skin is more likely to contain deoxygenated haemoglobin, and therefore deoxygenated haemoglobin is indicative of the amount of subcutaneous blood, or subcutaneous blood flow. Hence, monitoring deoxygenated haemoglobin gives an indication of the effectiveness of heat in the body being brought to the skin to be dissipated into the environment.

In a further aspect, the invention proposes a method for monitoring insufficient body heat dissipation in a subject, comprising the step of: raising an alert if an increase in temperature of the subject is not accompanied by a concurrent and sufficient increase in subcutaneous blood flow to a level pre-determined for the extent of the increase in temperature.

Preferably, the method further comprises the steps of: observing heart rate of the subject; and requiring a concurrent increase in heart rate before the alert is raised. This combines use of the heart rate, or pulse, temperature and subcutaneous blood flow (or alternatively the amount of subcutaneous blood) to give a non-diagnostic, preliminary indication of whether the subject should be seen by medical personal for a diagnosis. Where the method is used in an ear worn device such as a earbud or a earphone, susceptible people such as firemen, the infirmed, people in a state of comatose, drivers, prisoners can be monitored for their health condition in a convenient, non-invasive and a manner almost unnoticeable to other people.

More preferably, the method further comprises the steps of: observing heart rate variation of the subject; and requiring a concurrent reduction in the heart rate variation before the alert is raised.

In yet a further aspect, the invention proposes a folded flexible printed circuit board comprising: a substrate imprinted conductive lines to provide electrical circuitry; one or more portions of the substrate being relatively flexible portions; and one or more portions of the substrate being relatively rigid portions; wherein the substrate folded such that the relatively rigid portions define a space for accommodating electronic components on the substrate.

Printed circuit boards are relatively new, and provide the possibility of squeezing electrical circuitry into devices of challenging shapes. However, the tendency is to make flexible printed circuit boards as flexible as possible. The invention teaches in the opposition direction of providing rigidized portions in the flexible printed circuit boards. This rigidized portions can be folded to provide a functional shell, which provide protection for electronic components that are sensitive to contact and placed onto the flexible printed circuit boards, such as optical sensors, temperature sensors, piezoelectric components, pyroelectric components and so on. Moreover, providing space around sensitive components allows better drying of damp that might have crept into the component. Hence, the protection improves the efficiency and lifespan of such touch sensitive components.

Typically, the rigidity of the remainder of the substrate is provided by application of a layer of rigid material to the substrate. For example, the layer of rigid material is metal. In some cases, the electronic components include at least two heat detectors. Preferably, the at least two heat detectors are semiconductor temperature sensors.

In a yet further aspect, the invention proposes an earbud comprising: a bud for placement into an ear canal when the earbud is worn by a subject; and the folded flexible printed circuit board as described contained within the bud.

In a yet further aspect, the invention proposes a method for monitoring a subject of his body heat, comprising the steps of: obtaining an indication of the level of heat in the ear canal of the subject at the same time as obtaining the heart rate of the subject; and raising an alert if the heart rate is lower than a pre-determined upper threshold heart rate variation (HRV) for the level of heat in the ear canal. This invention provides the possibility of determining if the increase in heart rate is causing the subject any anxiety or sense of discomfort as indicated by a lower HRV.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows a comparative prior art example to an embodiment of the invention;

FIG. 2 is a schematic diagram of an embodiment of the invention;

FIG. 3 is a schematic diagram of the internal parts of the embodiment of FIG. 2;

FIG. 4 shows how the embodiment of FIG. 2 is put in use;

FIG. 5 shows how the embodiment of FIG. 2 is positioned in the ear;

FIG. 6 is a magnified drawing showing how the embodiment of FIG. 2 is positioned in the ear;

FIG. 7 shows a linear relationship model to which temperatures read by the embodiment of FIG. 2 may be fitted;

FIG. 8 shows how the gradient of the model of FIG. 7 may change;

FIG. 9 also shows how the gradient of the model of FIG. 7 may change;

FIG. 10 also shows how the gradient of the model of FIG. 7 may change;

FIG. 11 also shows how the gradient of the model of FIG. 7 may change;

FIG. 12 is a flowchart showing the deliberations during the operation of the embodiment of FIG. 2;

FIG. 12a shows a variation of the embodiment of FIG. 2 used to obtain the body temperature of a subject;

FIG. 13 is a variation of FIG. 12a;

FIG. 14 is a magnified drawing showing how a variation of the embodiment of FIG. 2 is positioned in the ear;

FIG. 15 is a drawing of the pinna, showing the concha;

FIG. 16 shows how a variation of the embodiment of FIG. 2 is used on the pinna shown in FIG. 15;

FIG. 17 shows a variation of the embodiment of FIG. 2 which is part of a hearing aid;

FIG. 18 shows a further variation of the embodiment of FIG. 2 which monitors hypothermia;

FIG. 19 shows yet a further embodiment;

FIG. 20 shows the heart rate of a person that may be observed by the embodiment of FIG. 19;

FIG. 21 is a schematic diagram of the internal parts of the embodiment of FIG. 19;

FIG. 22 shows how the embodiment of FIG. 19 is put in use;

FIG. 22a shows changes to the pulse of a person that may be observed by the embodiment of FIG. 19;

FIG. 22b is a flowchart show one of the possible methods of determining if a subject needs medical attention using the embodiment of FIG. 19;

FIG. 22c is a variation of the flowchart of FIG. 22b;

FIG. 23 shows an alternative to the embodiment of FIG. 19 from two opposite directions;

FIG. 24 shows another alternative to the embodiment of FIG. 19 from two opposite directions;

FIG. 25 shows yet another alternative to the embodiment of FIG. 19;

FIG. 26 shows yet another alternative to the embodiment of FIG. 19;

FIG. 27 shows yet another alternative to the embodiment of FIG. 19 from two different directions;

FIG. 28 shows yet another alternative to the embodiment of FIG. 19;

FIG. 29 shows a system which is an alternative to the embodiment of FIG. 19;

FIG. 30 shows a part of prototype made according to an embodiment of the invention;

FIG. 31 shows enlarged view of portions of the part of the prototype shown in FIG. 30;

FIG. 32 shows the part of prototype shown in FIG. 30 arranged to fit into an optical head of the prototype;

FIG. 33 shows the front casing of the prototype mentioned in FIG. 30;

FIG. 34 shows the prototype mentioned in FIGS. 30; and 19

FIG. 35 is a side-wise cross-sectional view of the prototype of FIG. 34.

### DETAILED DESCRIPTION OF EMBODIMENTS

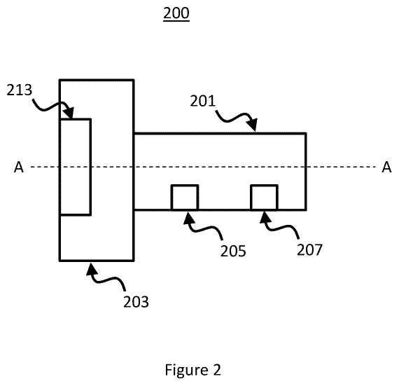

FIG. 2 shows an embodiment 200 of the present invention, which is a device 200 that may be inserted into the opening of an ear canal. The embodiment 200 comprises a plug 203, and an extension 201 or elongate member 201 extending from the plug 203. The user holds the embodiment 200 by the plug 203 when the user inserts the extension 201 into one of the user's ear canals.

The plug 203 has dimensions suitable for fitting into and closing the opening of the ear canal. Preferably, the plug 203 is made of a soft, deformable material such as rubber, silicon or some other kinds of polymers which can deform in order to squeeze into the opening of the ear canal and stay there securely. A good fit restricts or reduces flow of air into and out via the opening of the ear canal. Consequently, this reduces air exchange with the surroundings. The side of the plug 203 facing away from the extension 201 is installed with an LED (light emitting diode), which flashes if an alarm is raised when the user wearing the embodiment 200 is in risk of core temperature over-heating or a heatstroke.

Along axis AA of the extension 201 shown in FIG. 2 are placed two thermistors 205, 207. The thermistors measure temperatures of the air in the ear canal, in different locations, typically in Celsius or Fahrenheit. In other embodiments, thermocouples, any types of miniature thermometers or temperature monitors may be used instead of thermistors. The thermistors 205, 207 are spaced apart from each other along the extension 201. The positions of the thermistors 205, 207 on the extension 201 are pre-determined; the distance of each of the thermistors 205, 207 from the plug 203 is known, as well as the distance Δx between the thermistors 205, 207. Preferably, the thermistors 205, 207 are placed on the same side of the axis of the extension 201, such that the thermistors 205, 207 face roughly the same direction.

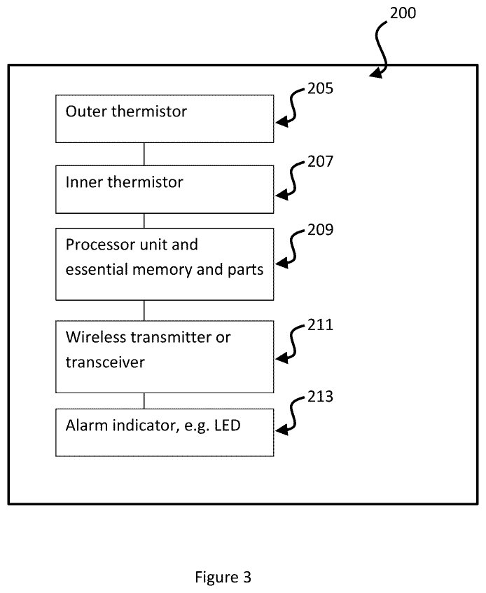

FIG. 3 schematically illustrates some parts of the embodiment 200 which include, besides the inner thermistor 207 (inner meaning further into the ear canal than the outer thermistor 205 when the embodiment 200 is worn) and outer thermistor 205 (outer meaning nearer the opening of the ear canal than the inner thermistor 207 when the embodiment 200 is worn), a processor and any required memory or parts for the processor to operate 209, a wireless transmitter or transceiver 211 and an alarm 213 to indicate overheating of the user wearing the embodiment 200, which is the LED in this embodiment 200. Optionally, the alarm also includes sonic functions to sound an alarm to the user or to people around the user. Furthermore, the alarm optionally includes a speaker (not illustrated) which plays a pre-recorded message into the ear of the user to warn him of his risk of over-heating, operating in a similar way as an earphone.

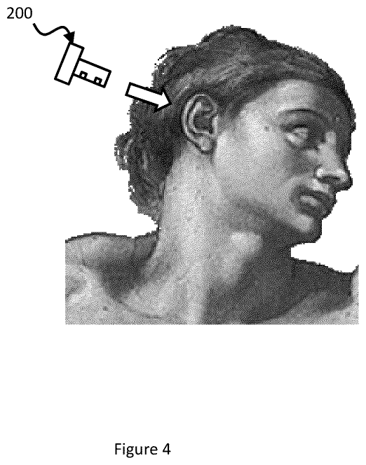

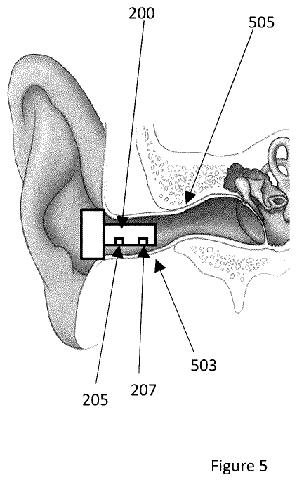

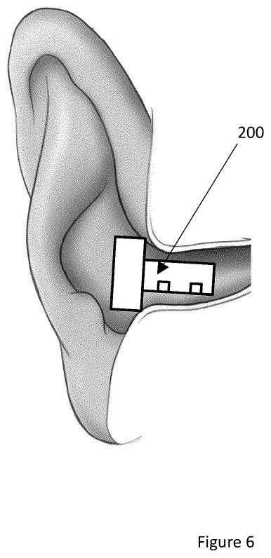

FIG. 4 shows how the embodiment 200 is inserted into the ear canal. Typically, the plug 203 is held by the user's fingers (fingers not illustrated) and the extension 201 is pointed towards the deep end of the ear canal. The plug 203 is then inserted to stop the opening of the ear canal. FIG. 5 is a cross-sectional diagram showing how the embodiment 200 is placed inside the ear canal. FIG. 6 is a magnified part of FIG. 5.

The deformable material which the plug 203 is made of has enough firmness such that the extension 201 is able to cantilever from the inserted plug 203 without resting on or contacting any part of the ear canal wall. When properly inserted, the extension 201 is centrically located in the ear canal and along the axis of the ear canal. On the average, the adult human ear canal extends from the pinna to the eardrum over distance of about 2.5 cm (1″) in length, and has a diameter of 0.7 cm (0.3″). Therefore, the diameter of the extension 201, when regarded axially, is preferably less than 0.7 cm in order to fit into most ear canals. More preferably, however, the diameter is equal to or less than 0.5 cm. This provides that the extension 201 is narrower than most ear canals, increasing the possibility that the sides of the extension 201 are not in contact with ear canal wall. As a result, the user feels only the plug 203 in the opening of the ear canal and possibly not the extension 201. If so, this provides that the embodiment 200 is comfortable enough for the user to wear the embodiment 200 over a long period of time. More importantly, this provides that thermistors 205, 207 measure temperatures of the air in the ear canal and not temperature of the ear canal wall or tissue.

Yet more preferably, the extension 201 has an even smaller diameter at about 0.3 cm, which may provide just enough structural support for the location of the thermistors 205, 207 on the extension 201, and the bluntness of a 0.3 cm extension 201 prevents the tip of the extension 201 from piercing the skin of the ear canal.

The typical ear canal is not a straight passageway. As one can see in FIG. 5, the typical ear canal has two bends, labelled 503 and 505. The first of the bends 503 is rather near the opening of the ear canal. The distance of the first bend 503 from the opening of the ear canal is usually slightly more than 1 cm in most people. Hence, the extension 201 is preferably short enough, at 1 cm or less, to avoid contact with this bend 503.

As the plug 203 generally prevents airflow exchange between ambient air and the air in the ear canal, in a state of equilibrium the temperature of the restricted air in the plugged ear canal is largely due to heat emanating from the core of the body.

Body heat is generated in the body and carried to the skin by flow of blood to be dissipated in the form of bodily radiation and by sweat. Large blood vessels are found deep in the body, carrying most of the warm blood in the body. Some blood in the head near the ear flows towards the pinna, through smaller blood vessels. The skin around the ear and structure of the pinna provides a large surface in which many small capillary blood vessels dissipate heat away from the body quickly. Hence, the temperature of the air in the ear canal near the opening is cooler than the temperature of the air deeper in the ear canal even if the ear canal is plugged. Furthermore, even when temperature at the opening of the ear canal is momentarily higher than the deeper parts of the ear canal, blood in the capillary blood vessels tends to absorb the heat and carry the heat to be dissipated at the skin, thereby cooling the temperature at the ear canal opening. In equilibrium, a relatively stable temperature gradient may be observed.

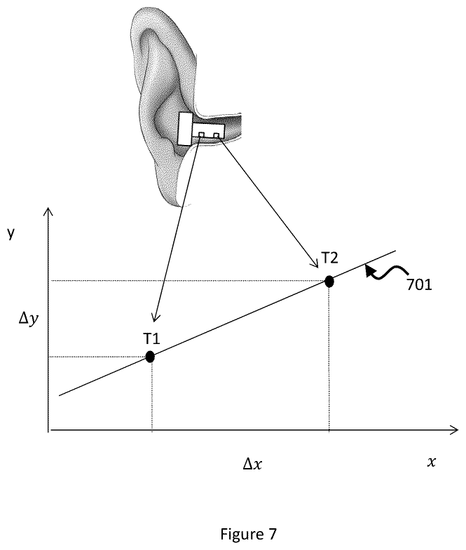

FIG. 7 is a chart showing temperature on the vertical y-axis, and distance in the ear canal on the horizontal x-axis. As illustrated in FIG. 7 and explained above, the temperature of air in the ear canal nearer the opening is lower than the temperature of air deeper in the ear canal. This creates a natural temperature gradient 701 in the ear canal. The thermistors 205, 207 are used to observe this temperature gradient.

As the skilled man knows, temperature gradient is a physical quantity describing the direction and the rate of temperature change. Temperature gradient may be expressed in units of degrees (e.g. Celsius) per unit length, its SI unit being kelvin per meter (K/m), or as expressed as dQ/dt, the rate of heat transfer per second.

However, for simplicity, the temperature gradient in this embodiment 200 is merely expressed as the spread of temperature in the ear canal, Δy, over the physical distance between the inner thermistor 207 and the outer thermistor 205, Δx.

Therefore, assuming the temperature gradient, VT, to be linear, it is expressed as follows:

$$∇ T = \frac{Δ y}{Δ x} \qquad ( 1 )$$

As the thermistors 205, 207 are spaced apart along the axis of the extension 201, each of the thermistors 205, 207 measures the temperature of the air in the ear canal in their different, respective locations in the ear canal. Accordingly, temperature which each of the thermistors may detect is different from temperature detected by the other thermistor.

The temperature of the air in the part of the ear canal nearer the opening, measured by the outer thermistor 205, is labelled T1 in the drawings. The temperature of the air in the deeper part of the ear canal, measured by the inner thermistor 207, is labelled T2 in the drawings. In FIG. 7, the temperature gradient of the air in the ear canal is shown increasing from T1 to T2.

The temperatures of the air in the ear canal are unlikely to be the same as the actual temperature of the body. For example, the body may be running a fever at 39° C. but T2 may just be a cooler 32° C. In most temperate and tropical places, the air in the ear canal is normally warmer than the ambient temperature but cooler than the actual body temperature. This is partly because of the relatively lower capacity of air than blood to take up heat, as well as the continuous flow of blood along the ear canal which absorbs away any amount of heat causing the temperature of the air in the ear canal to be greater than the body temperature. That absorbed heat is dissipated by the skin of the pinna to the surroundings outside the ear canal.

Despite the difference between the temperatures of the air in the ear canal and the actual body temperature, the embodiment 200 is able to determine that the user has a life-threatening increase in core body temperature by monitoring for steepening of the temperature gradient. In this way, the embodiment 200 does not require exact measurement of body temperature. This also relieves the need to place the thermistors in precise locations along the ear canal. People with shallower or longer ear canal may use the embodiment to monitor their body heat status, as a temperature gradient may be obtained and observed for changes whether the extension is inserted deeply into the ear canal or not.

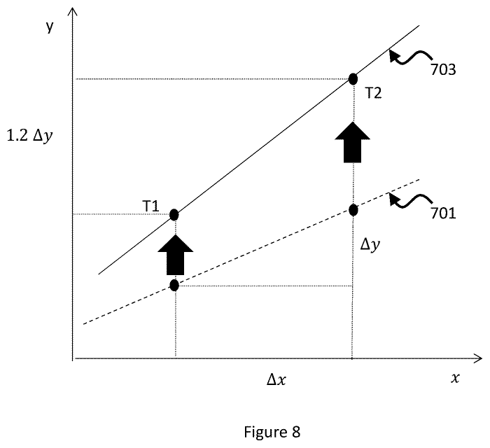

FIG. 8 shows how change in the temperature gradient can be used to determine if the user is overheating. If the core temperature of the user has increased suddenly, such as in a case of an imminent heatstroke, heat inside the body will be generated more quickly than heat may be dissipated by the skin. As a result, the temperature of the air monitored by the inner thermistor 207 in the deeper part of the ear canal, T2, increases. The temperature of the air monitored by the outer thermistor 205 in the part of the ear canal nearer the opening, T1, also increases but to a lesser extent than T2, partly due to the heat dissipative function of the nearby blood, pinna and skin. Eventually, equilibrium is reached and a new temperature gradient 703 having a greater value than the original Δy/Δx, and which is steeper, is observed.

If the extent of the change of the temperature gradient 701 into the new temperature gradient 703 is more than a threshold level, such as 20% more than the original Δy/Δx, the embodiment 200 raises an alarm indicating that the user might be in imminent danger of a heatstroke. In other words, if the new temperature gradient 703 has a value of 1.2×Δy/Δx, as illustrated in the chart of FIG. 8, an alarm that the user is over-heating is raised using the alarm 213.

“20%” is an arbitrary example of a threshold given here, and the actual threshold can be determined finally by the manufacturer of a product embodying the invention. Instead of 20%, the actual threshold can be determined by making statistical observations on people, or even other methods, but these are beyond any need of elaboration for the scope of this description.

Also, 20% may refer only to the amount of change of the temperature range Δy, as read between the outer thermistor 205 and the inner thermistor 207. That is, if the original T2 is 30° C. and T1 is 28° C., the 20% increase means a 20% increase on the range of 28° C. to 30° C., or 0.2×2° C., which is just about 0.4° C. That is, if Δy increased by about 0.4° C., the alarm is raised. Therefore, a rise of 20% in the temperature gradient in the ear canal air does not necessarily translate to a 20% increase in actual body temperature.

In practice, after the user puts on the embodiment 200, the temperatures of the air in the respective locations in the ear canal, i.e. T1 and T2, are measured with the opening of the ear canal plugged. As soon as T1 and T2 have stabilised, an initial temperature gradient 703 is observed. It does not matter whether the user's normal body temperature is naturally higher or lower than the theoretical normal body temperature. The exact temperature of different normal, healthy individuals actually varies from person to person, and is not always 36.9° C. Subsequently, the embodiment 200 monitors for significant changes in the temperature gradient to determine whether there is a risk of an imminent heatstroke.

As there is no need to obtain the exact temperature of the user's body, the embodiment 200 does not require calibration for interpreting the temperature gradient of the ear canal air into actual body temperature. Not having to operate with exact, accurate temperature reduces the sensitivity requirement of the embodiment 200, making the embodiment 200 robust, not overly-delicate and suitable for deployment in rugged use.

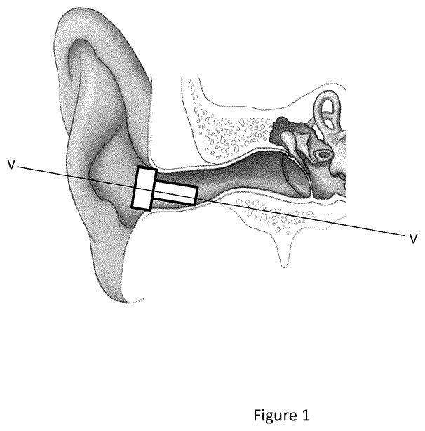

In contrast, if only one thermistor were used to monitor the user's risk of heatstroke, exact body temperature would have to be read and the thermistor would have to be placed deep into the ear canal, to be as near the ear drum as possible. This is because the ear is largely a heat dissipating organ, and the outer ear can be much cooler than the core of the body. This is also the reason why the tympanic infrared thermometer needs to have a line of sight to the tympanic membrane for accurate measurement. An illustration of an ear-wearable tympanic infrared thermometer, as a comparative example, is shown in FIG. 1. The tympanic infrared thermometer is shown off-alignment to the tympanic membrane, pointing instead in the line XX, which prevents reading of accurate body temperature.

Accordingly, the embodiment 200 moves away from the conventional teachings of measuring exact body temperature in order to monitor risk of heatstroke, and also does not require line of sight to the tympanic membrane, which is unlike the tympanic infrared thermometer. Hence, any misalignment of the present embodiment 200 to the central axis of the ear canal is unlikely to reduce the effectiveness of the embodiment 200 to raise an alarm to a risk of heatstroke.

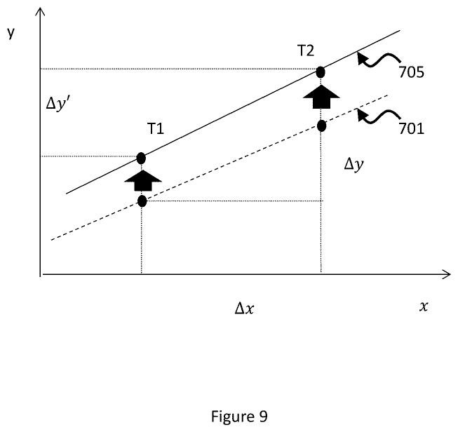

FIG. 9 shows how a user's core body temperature can rise in some circumstances, even though the user is not in danger of heatstroke. Such circumstances must be distinguished from other circumstances which carry a risk of heatstroke. When the user wearing the embodiment 200 engages in strenuous activities, T2 increases due to the rise in core body heat. T1 also increases but only to a smaller extent as heat is dissipated effectively at the pinna by the skin, in the form of radiation and by sweating. In other words, heat is dissipated away fast enough. Accordingly, it is observed that there is little change in the magnitude of Δy′ compared to Δy, and the new temperature gradient 705 in this case has not steepened very much from the original temperature gradient 701. As the slight change in temperature gradient does not reach the extent of a pre-determined threshold, the alarm to warn of a heatstroke is not raised.

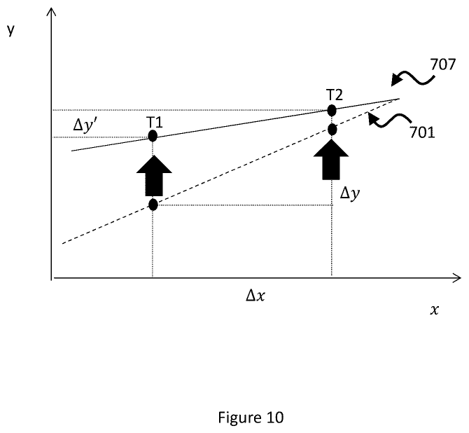

FIG. 10 shows a situation in which the temperature gradient changes to become gentler instead of steeper. This occurs when the user steps into an environment where the ambient temperature is hotter from another environment where the ambient temperature is cooler and, as a result, his body heat is not as effectively dissipated as in the earlier environment. However, the user remains able to tolerate the surrounding heat because his core body temperature has not increased much. As shown in FIG. 10, T1 increases significantly and T2 increases just a little or does not change, and magnitude of Δy′ of the new temperature gradient 707 reduces compared to Δy of the original temperature gradient 701. In this case, because the temperature gradient has become gentler, the alarm to warn of a heatstroke is not raised.

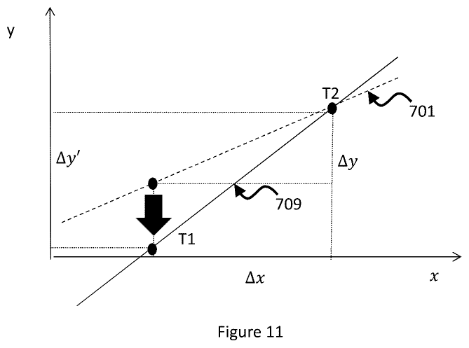

Preferably, to determine that there is an imminent danger of heatstroke further requires both the two thermistors to detect a rise in the temperatures of the air in their respective locations in ear canal. In other words, there is a positive increase of both T1 and T2 besides steepening of the temperature gradient. FIG. 11 illustrates this case. This is to avert false alarm caused by a steepening of the temperature gradient which is due only to reduction in T1. Such a reduction of T1 may be caused by the plug 203 not stopping airflow into and from the ear canal sufficiently, and cold ambient air interacts with the air in the ear canal near the opening, or may be caused simply because the ambient temperature is extremely cold. When T1 decreases but T2 does not change, a new temperature gradient 709 which is steeper will be observed. This is because Δy′ of the new temperature gradient 709 is greater than Δy of the original temperature gradient 701. Therefore, to distinguish this harmless steepening of the temperature gradient from the kind of steepening which accompanies a heatstroke, the alarm to warn of a heatstroke is not raised if T1 and T2 did not both increase.

Optionally, the temperatures measured by both thermistors 205, 207 are sent wirelessly to a remote computing device or server to deduce the temperature gradient. This is to reduce data processing in the embodiment 200 as much as possible, especially if the embodiment 200 is worn by a user who is fireman in a hot, fire rescue situation. Less tasks for the processor to execute means the embodiment 200 is able to operate more efficiently and with less energy consumption. Alternatively, the temperatures measured by both thermistors 205, 207 are compiled into a temperature gradient by a processing device inside the embodiment 200. Information on the threshold of temperature gradient change is pre-stored in the processor's memory. The processor is thereby able to check at any time if the extent of change in the temperature gradient has reached the pre-determined threshold.

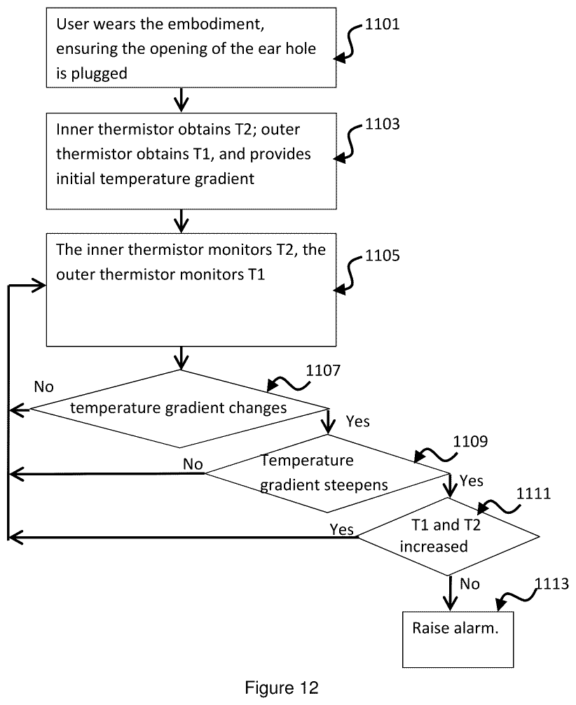

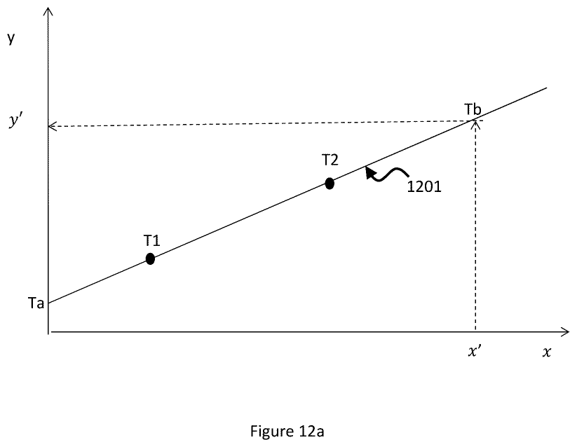

FIG. 12 is a flowchart corresponding to the situations illustrated in FIG. 8 to FIG. 11, showing how the embodiment 200 is used to determine if the user is in imminent danger of heatstroke.

In step 1101, the user inserts the embodiment 200 into his ear. The plug 203 stops air in the earhole from mixing with ambient air. In step 1103, the outer thermistor 205 measures T1 in a part of the ear canal nearer the opening of the ear canal, while the inner thermistor 207 measures T2 in a deeper part of the ear canal. A temperature gradient 701 is observed when the temperature of the air in the ear canal has stabilized. At this point, as the user has just put on the embodiment 200 into his ear, his body temperature at this very instant is assumed to be normal, i.e. typically deemed 36.9° C. This is because, if the user is a fireman about to fight a fire, he is unlikely to be running a fever already. Hence, the initial condition of the user is taken to be the reference against which he will be monitored for deviation therefrom. In other words, whatever temperature gradient is observed in the ear canal when the user first puts on the embodiment 200 will be deemed the reference temperature gradient or original temperature gradient 701, against which gradient change is observed, compared and evaluated. The original temperature gradient 701 is obtained afresh every time the user wears the embodiment 200 anew.

At step 1105, the thermistors 205, 207 monitor the temperatures of the air in the ear canal continuously. If no change in temperature gradient is observed, at step 1107, the thermistors 205, 207 simply continue, at step 1105, to monitor the temperatures of the air in the ear canal. If a change in the temperature gradient in the ear canal is observed, at step 1107, then the next step is to determine, at step 1109, if the temperature gradient has steepened compared to the original temperature gradient 701, or has become gentler.

If it is determined, at step 1109, that the temperature gradient has not steepened sufficiently or has become even gentler in the direction of T1 to T2, the thermistors 205, 207 returns to monitoring the temperatures of the air in the ear canal, at step 1105. There is no need to raise any alarm.

On the other hand, if it is determined, at step 1109, that the temperature gradient has significantly steepened in the direction of T1 to T2, reaching the pre-determined threshold, the next step is to determine if both thermistors 205, 207 observe an increase in temperature. That is, whether T1 and T2 have both increased. This ensures that the false alarm as described in FIG. 11 is not raised. Therefore, if it is determined, at step 1111, that both T1 and T2 have increased, an alarm is raised, at step 1113, to warn that user that he is at risk of a heatstroke.

Optionally, in some embodiments, even if it is determined that only T2 has increased, but T1 has remained constant, an alarm is also raised to warn that the user is having a risk of heatstroke. This is because an increase in T2 is probably due to increase in core body temperature despite not being accompanied by an increase in T1.

Optionally, if the steepening of the temperature gradient is caused by an increase in T2 (increase in core temperature) but also by a decrease in T1 (probably due to cooler ambient temperature), a stricter threshold may be applied, such as by requiring a 25% increase in the gradient instead of the 20% (given as example above). A higher threshold helps to ensure that there is a real risk of heatstroke before an alarm is raised, and that the significant steepening of the temperature gradient is not caused largely by colder ambient temperature.

If it is determined that requiring an observation of a steepening of the temperature gradient is caused only by a decrease in T1, as described in FIG. 11, then the cause of the steepening of the temperature gradient is due to a cooler ambient environment, and no alarm is raised.

Although embodiments have been described which does not require the exact temperature of the user to be known to raise a heat stroke alarm, it is nevertheless possible in some embodiments to determine the exact body temperature of the user. FIG. 12a shows such an embodiment, in which the temperature gradient 1201 can be used to make an extrapolation to determine the actual temperature, y′, of the user. In FIG. 12a, the ambient temperature is labelled Ta. The user's actual temperature is labelled Tb. Ta and Tb are two points which forms a linear relationship. T1 and T2 are the temperatures of specific locations in the ear canal, as observed by the thermistors 205, 207 and are in line with the relationship between Ta and Tb. Mathematically, they may be expressed as follows:

T1=f(Ta,Tb) (2)

T2=f(Ta,Tb) (3)

Therefore, it is possible to deduce Tb from the relation as supposed by the model, where the tympanic membrane is assumed to be in position x′ in the ear canal. Position x′ can be established for each individual user using any measurement methods, or may simply be estimated.

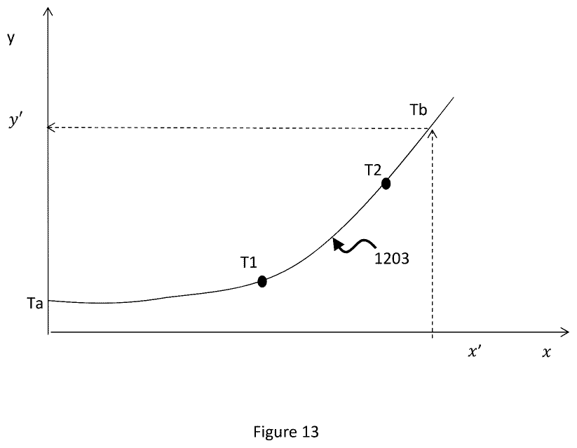

FIG. 13 is a variation of FIG. 12a. While FIG. 12a uses a linear relationship model to predict Tb, FIG. 13 shows the relationship model to be a curve 1203 that extends exponentially. As with the case in FIG. 12a, T1 and T2 are measured by the inner thermistor 207 and the outer thermistor 205, and the model is used to obtain Tb. Any other relationship model can be used. The specific relationship model to use is a choice for the manufacturer of the product embodying the invention to make, which may depend on the brand and make of the thermistors. It is possible to fit the temperature of the air in the ear canal as read by the two thermistors to a pre-selected curved model. Preferably, it is possible to provide more than two thermistors on the extension to read and plot a curved model, i.e. provide at least three points of temperature in the ear canal which spread out in a curve model such as that in FIG. 13a (not illustrated).

Regardless of the choice of model, be it a linear one as shown in FIG. 12a or a curved one as shown in FIG. 13, the relationship can be calibrated to more accurately predict the user temperature. For example, the initial temperature gradient can be calibrated to the user's temperature when he first wore the embodiment 200, by assuming that the temperature is 36.9° C. This would be a one-point calibration. Henceforth, any change in the temperature gradient relies on the calibration to predict the temperature of the user. Specific details of convention calibration methods are well known and do not require elaboration here. In such embodiments in which the actual body temperature of the user is measured, it is an option to raise the alarm to warn of the user being at risk of heatstroke when the body temperature of the user has risen and reached a specific threshold temperature, such as 38° C., instead of relying on an extent of change in temperature gradient to raise the alarm.

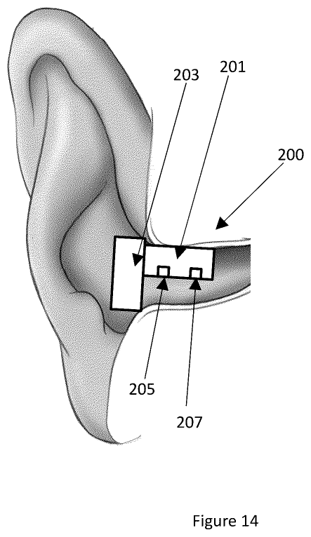

FIG. 14 shows a variation of the embodiment 200, the variation being in the position of the extension 201 on the plug 203. The extension 201 is located on the plug 203 in such a way that when the plug 203 is fitted properly into the opening of the ear canal, the extension 201 is positioned in the ear canal eccentrically. One side of the extension 201 touches the ear canal wall. To ensure that the thermistors 205, 207 do not measure temperature of the ear canal wall and only measure the air in the ear canal, the thermistors 205, 207 are placed on the other side of the extension 201 which is not in contact with the ear canal wall. Advantageously, this allows the user to feel the presence of the extension 201, which lends a sense of security to users who would prefer to know by touch that the extension 201 has been positioned properly.

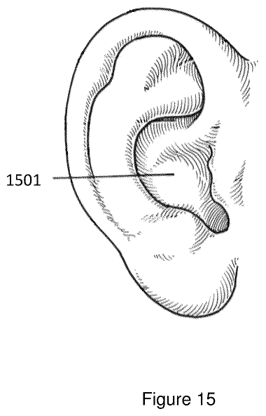

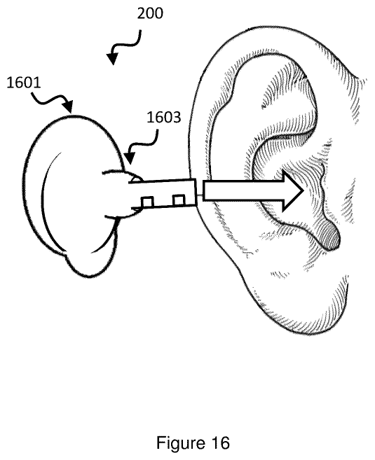

In a preferred variation of the embodiment 200, illustrated in FIG. 16, a part 1601 of the plug 203 has a shape which is moulded to the shape of the concha of an ear of a particular user. FIG. 15 is an illustration of the outer human ear. The concha 1501 is the part of the ear which is a depression just around the opening of the ear canal. The concha has a unique, asymmetrical shape, and varies from user to user. This part 1601 of the plug is usually made of a hard, non-deformable material, such a hard thermoset plastic like Bakelite, glass or fibre glass. The part 1603 of the plug which is to stop the opening of the ear canal is made of a deformable material which can deform to squeeze into the opening of the ear canal. Hence, the plug of this embodiment is made up of a hard, outer part 1601 for the concha, and a soft, inner part 1603 for the opening of the ear canal. Fitting a part of the embodiment 200 to the concha 1501 provides that the position of the plug 203 in the concha and the position of the extension 201 within the ear canal is the same every time the user wears the embodiment 200. This further ensures that the extension 201 is arranged properly in the ear canal and that the thermistors 205, 207 do not touch the ear canal wall.

In another embodiment which is not illustrated, the embodiment is placed within an earphone which is capable of receiving communication information wirelessly such as via Bluetooth™. Such an earphone can be worn by every member in a team of firemen to engage in a dialogue with each other and to coordinate themselves during a fire rescue. If the embodiment determines that any one of the firemen is likely to suffer from a heatstroke, the alarm raised includes an audio message sent to the earphones worn by all the team members.

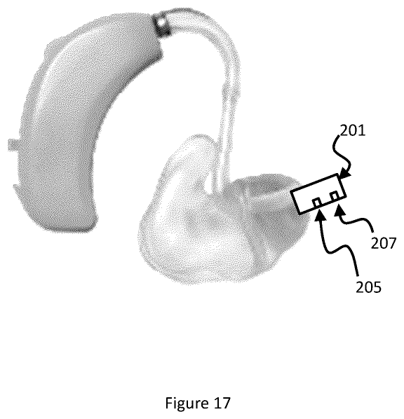

FIG. 17 shows another embodiment 200 which is a hearing aid fitted with an extension 201 having the thermistors 205, 207 as described in the aforementioned embodiments. As elderly people tend to wear a hearing aid throughout the day, this embodiment 200 allows elderly people to be monitored continuously for increase in body temperature without the elderly people feeling bothered by it. This embodiment is particularly helpful in nursing homes in which private nursing attention is spread thin.

Accordingly, the embodiments include a method for determining a state of over-heating or a risk of over-heating of a subject, i.e. user of the embodiments, comprising the steps of: obtaining the temperature gradient 701 of an ear canal of the subject; detecting a change in the temperature gradient; and determining a state of over-heating or risk of over-heating if the change in temperature gradient is beyond a pre-determined threshold level.

Typically, a subject who is considered as over-heated means his core temperature has risen beyond an acceptable normal level. This does not mean that the subject is already delirious or has suffered a heatstroke, as that would be quite apparent to anyone around him. In most situations, the meaning of the subject over-heating means that the subject's core temperature has raised so high and his ability to dissipate the heat is so bad that he is in danger or risk of suffering injury and immediate treatment should be given to prevent injury, i.e. a stage before serious injury or permanent damage has set in.

Nevertheless, the exact definition of over-heating can be established by each manufacturer of a specific product containing an embodiment of the invention. Over-heating could, for example, be defined to mean that the user has already entered into a state of delirium or heatstroke. Although this would be a less useful threshold as the damage has already set in, a product which detects such a stage may still find some use in setting off a heightened alarm, such as a louder alarm siren from the embodiment than the alarm siren for the subject merely having a risk of imminent heat injury. The heightened alarm indicates greater urgency.

Furthermore, the embodiments include a device 100 for observing temperature in an ear canal of a subject, comprising a plug suitable for restricting air flow through the opening of the ear canal; a first thermistor 205 arranged to measure the temperature of the air in a first position in the ear canal; and a second thermistor 207 arranged to measure the temperature of the air in a second position in the ear canal; the second location being deeper in the ear canal than the first location.

While there has been described in the foregoing description preferred embodiments of the present invention, it will be understood by those skilled in the technology concerned that many variations or modifications in details of design, construction or operation may be made without departing from the scope of the present invention as claimed.

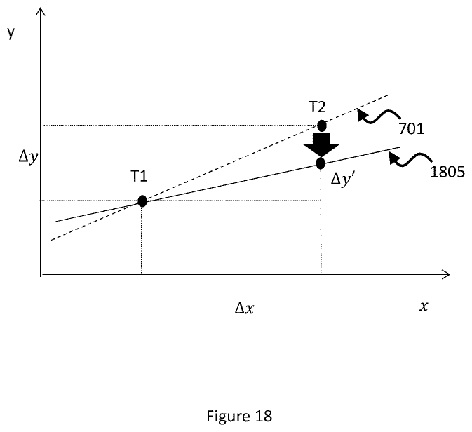

For example, instead of monitoring for risks of over-heating, FIG. 18 shows how the invention may be used in an embodiment which monitors hypothermia, which is a medical emergency that occurs when the body loses heat faster than it can produce heat. In this case, the body turns cold instead of running a fever. In the example given in FIG. 18, the inner thermistor detects that T2 has dropped significantly, which the outer thermistor detects little or no change. The temperature gradient 1805,

$$\frac{Δ y}{Δ x} ,$$

becomes significantly gentler,

$$\frac{Δ {y}^{′}}{Δ x} .$$

If the temperature gradient 1805 becomes gentler by a certain percentage that exceeds a pre-determined threshold, the alarm is raised to warn of hypothermia. This embodiment is useful for monitoring people who are in cold conditions, such as deep sea divers.

Furthermore, although the user has been described as a person, the embodiments may be applied to animals that require heatstroke monitoring, such as race horses. A horse can be inserted with an embodiment dimensioned and shaped to fit into the horse's ear.

Furthermore, although the thermistors 205, 207 have been described as placed on the same side of the axis of the extension 201, such that the thermistors 205, 207 face roughly the same direction, it is possible that the thermistors 205, 207 face opposite directions on the extension 201 which cantilevers from the plug 203. As long as the thermistors 205, 207 do not contact the ear canal wall, each is able to read the temperature of the air in respective location in the ear canal.

Furthermore, although two thermistors arranged on an extension extending from a plug has been described, variations of the embodiments which include two thermistors, each arranged on a separate extension, each of the extensions extending from the plug and to be inserted into the ear canal, are within the contemplation of this description (not illustrated). In such an embodiment, the first one of the thermistors is arranged on one of the extensions to be in the ear canal but nearer to the opening of the ear hole than the other thermistor, and the other thermistor is arranged on the other one of the extensions to be deeper in the ear canal than the first one of the thermistors.

Furthermore, although the change of temperature gradient in the ear canal has been described as change in the slope of a linear gradient, it is possible that the change may be that of a linear line to a curved line, in which case more than two thermistors are arranged on the extension. There can be as many thermistors as possible on the extension to observe a non-linear, curved temperature gradient. The curve may be exponential, sigmoid or logistic curve, or any other model as the manufacturer of an embodiment deems best suited.

### Further Embodiments

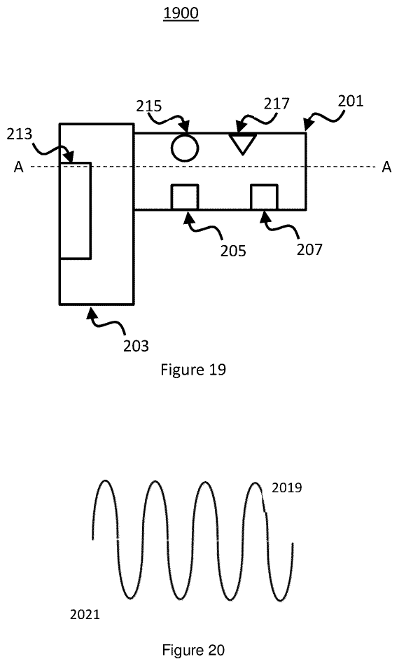

FIG. 19 shows another embodiment 1900, one which includes an improvement over the afore-described embodiments. The embodiment 1900 comprises a plug 203, and an extension 201 or elongate member 201 extending from the plug 203.

FIG. 19 also shows how, along an axis AA of the extension 201, are placed a light emitter 215 and an optical sensor 217. Together, the light emitter 215 and the optical sensor 217 operate as a PPG sensor (photoplethysmography). The light emitter 215 is typically a light emitting diode (LED), not to be confused with the afore-mentioned LED for indicating an alarm, and the optical sensor 217 is typically a photodiode. The light emitter 215 is positioned to emit light into the wall of the ear canal and into ear tissue. The optical sensor 217 is positioned to detect any part of that light that has been transmitted, scattered through and emerged from the ear tissue.

Although not shown in the schematic drawing, the skilled reader would appreciate that the light emitter 215 and the optical sensor 217 face such different directions and are so spaced apart, or that emission of the light emitter 215 is so directed, that stray, sidewise emission from the light emitter 215 does not reach or affect the optical sensor 217. The ways to provide such an arrangement is known to the skilled reader and is not given elaboration here.

Typically, the extension 201 has such dimensions and is so located on the plug 203 that, when the plug 203 is fitted properly into the mouth of the ear canal, the extension 201 extends therein eccentrically of the ear canal axis. To bring about this eccentric position, the axis AA can be seen in FIG. 19 as being off-centred on the plug 203. Furthermore, the extension 201 has a diameter small enough to provide that the extension does not fill up the ear canal. Therefore, the extension 201 has a side that is further away from the axis of the ear canal, such that any component installed on this side of the extension is more likely to contact the ear canal wall. Accordingly, the extension has another side that is nearer the axis of the ear canal, such that any component that is installed on this other side is less likely to touch the ear canal wall.

The light emitter 215 and the optical sensor 217 are placed on the side of the extension 201 that is likely to touch the ear canal wall. This could urge the light emitter 215 and the optical sensor 217 against the ear canal wall. Consequently, this increases the chance of the light emitter 215 emitting directly into ear canal tissue, and of the optical sensor 217 detecting mainly light that has transmitted through ear tissue.

The electromagnetic frequency and wavelength of the light emitter's emission is within the absorption spectrum of blood. Light of a wavelength within this spectrum that has transmitted through tissue of the user gives a pulsating signal, corresponding to the flushing of blood as pumped by the heart.

FIG. 20 schematically illustrates the pulsation. The peaks 2019 in the signal represent moments when the heart is relaxed and the ear tissue is relatively depleted of blood, such that more light transmits through the ear tissue to reach the optical sensor 217. The troughs 2021 in the signal represent moments when the heart has contracted and ear tissue is relatively full of light absorbing blood, such that less light transmits through the ear tissue to reach the optical sensor 217. By monitoring this pulsation, the PPG sensor obtains the heart rate, or pulse, of the user.

Preferably, like the embodiments mentioned before, the thermistors 205, 207 do not measure the temperature of the ear canal wall but only measure the temperature of the air in the ear canal. To bring about this effect, the thermistors 205, 207 are placed on the side of the extension that is less likely to touch the ear canal wall.

The distance into the ear canal over which the temperature gradient is observed is largely the same even between different people. The temperature gradient is a function of only a short distance into the ear canal and not for the entire length of the ear canal. The short distance is between an inner position where the core body temperature may be detected and an outer position at the mouth of the ear canal, which is cooled by heat dissipation at the outer ear. Hence, it is largely the physics of heat exchange that determines the depth of the temperature gradient rather than the size of a person or the size of his ear canal.

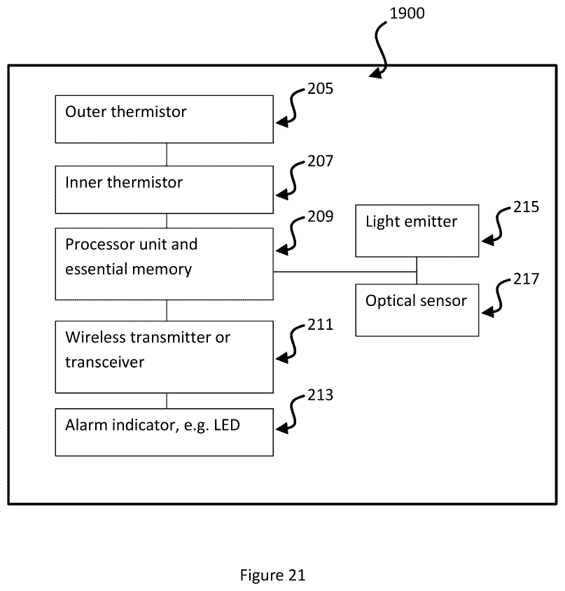

FIG. 21 schematically illustrates some functional parts of the embodiment 1900 of FIG. 19. FIG. 21 is similar to FIG. 3 but includes the light emitter 215 and the optical sensor 217.

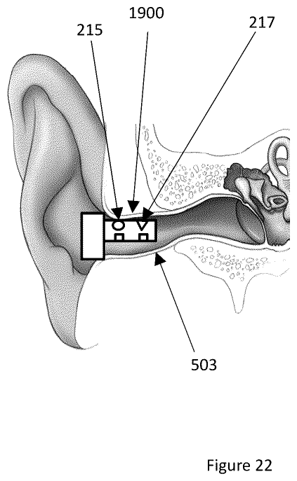

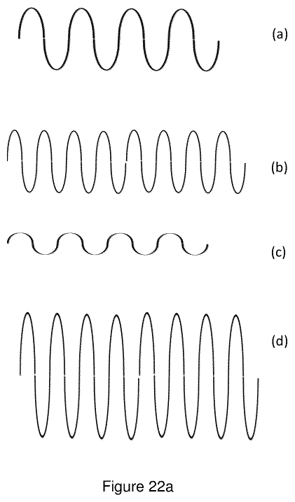

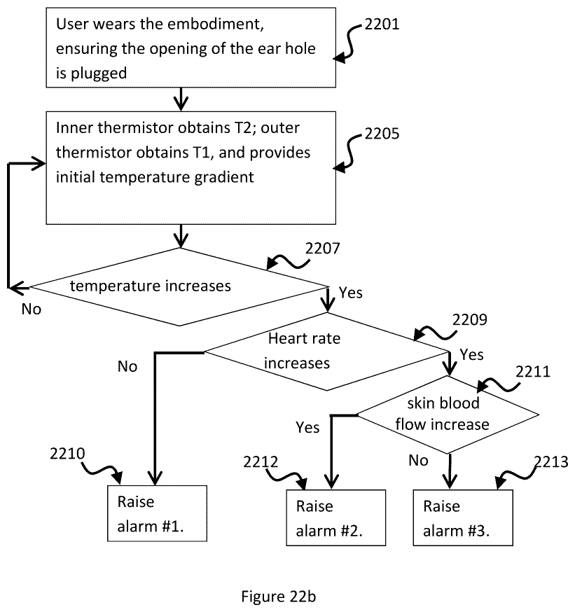

FIG. 22 shows how the embodiment of FIG. 19 is inserted into the ear canal before the ear canal's first bend 503, with the light emitter 215 and the optical sensor 217 in contact with the wall of the ear canal.

The embodiment 1900 of FIG. 19 also provides a possibility of observing the efficiency of the user's body heat dissipation. By observing from the ear canal the magnitude of the pulse, a rough indication of the extent of expansion and contraction of sub-skin blood vessels can be obtained. For example, changes in amplitude between individual heart beat signals, or changes in their graphical areas (area under the curve) or changes in their spread, can each or in combination be taken as an indication of a change in the amount of blood in the ear tissue. By extension, this may be taken as indicative of a change in the subcutaneous blood level.

Most parts of the outer ear and the ear canal are suitable locations to observe subcutaneous blood, as there are no big arteries, veins or organs inside these parts that could interfere with the absorption of transmitting light by subcutaneous blood.

A greater amount of subcutaneous blood is indicated by greater absorption of light transmitting through the ear canal tissue. Hence, a pulse with smaller peaks 2019 might mean more subcutaneous blood. In turn, more subcutaneous blood may be taken to indicate that the user's body is transporting heat to the skin for dissipation more effectively. However, the skilled reader would note that this is only a rough indication, as there are other factors that may affect the amplitude of the pulse, such as weakening of the heart. Accordingly, the embodiment does not make any diagnosis or conclusion, but may merely suggest a need for medical attention.

Therefore, an increase in body heat accompanied by an increase in pulse magnitude suggests poor heat dissipation. The situation is even more serious if this is further accompanied by an increased heart rate.

FIG. 22a illustrates how the heart rate of the user may change if the body has abnormal temperature. FIG. 22a drawing (a) shows the heart rate of the user at the start of the monitoring, or when he is calm and is not unwell. If the user's body temperature increases or if the user gets excited, the frequency of the heart rate increases as illustrated in drawing (b).

Drawing (c) illustrates a smaller pulse magnitude, which indicates an increase in the user's subcutaneous blood flow. This is because less light transmits through the user's ear canal tissue due to greater absorption of the light caused by more subcutaneous blood.

If the user has reduced blood flow in the skin, and if the user's heart rate has increased at the same time, then the frequency of the pulse increases as well as the pulse magnitude, as illustrated in drawing (d).

If the condition of drawing (d) is accompanied by an observation of increased bodily heat, such as when a change in the temperature gradient as illustrated in FIG. 8 is observed, an alarm 213 is raised to alert the user that he may be having a heat stroke.

FIG. 22b schematically illustrates a method for monitoring the heat condition of a user. Firstly, the user inserts a suitable embodiment into his ear canal, at step 2201. The inner thermistor and the outer thermistor then observe the temperature gradient in the ear canal, at step 2205.

Subsequently, if it is decided that there is no change in the temperature gradient, indicating that the user's body temperature has not changed, at step 2207, the method repeats the step of just observing the temperature gradient in the ear canal, at step 2205. If, on the other hand, it is decided, at step 2207, that the inner thermistor and the outer thermistor observe a change in the temperature gradient in the ear canal in such a way that indicates a rise in the user's body temperature, the method then checks if there is a concurrent increase in the heart rate of the user.

Then, if it is decided that there is no concurrent increase in the heart rate of the user, at step 2209, the method raises a first type of alarm, at step 2210, merely indicating that the user's body temperature has increased. If, on the other hand, it is decided that there is a concurrent increase in the heart rate of the user, at step 2209, the method then checks if there is a concurrent increase in the flow or amount of subcutaneous blood, at step 2211.

Then, if it is decided that there is a concurrent increase in the flow or amount of subcutaneous blood, at step 2211, the method raises a second type of alarm, at step 2212, merely indicating that the temperature has increased along with the heart rate. This is because there is generally no need to raise an alarm about the increase in subcutaneous blood, as that indicates a heathy possibility of improved heat dissipation at the skin.

If, on the other hand, it is determined that there is no concurrent increase in the flow or amount of subcutaneous blood, at step 2211, the method then raises a third type of alarm, at step 2013, indicating a possibility that the body temperature has increased along with an increase in heart rate, but without an increased rate of heat dissipation from the user's skin.

Optionally, the third type of alarm is raised, at step 2013, if it is determined that there is a concurrent reduction in the flow or amount of subcutaneous blood, at step 2211.

The skilled man would understand that the flowchart in FIG. 22b is just an example out of many other possible examples, and different orders of observing the temperature, heart rate and skin blood flow are within the contemplation of the invention.

In a further embodiment, it is possible for the user's efficiency at dissipating heat to be evaluated by measuring the ratio between arterial blood volume as and the venous blood volume in ear canal tissue. This method gives a rough indication of the extent that heat is assumed to be retained by venous blood, relative to the extent that heat is carried by capillary arterial blood moving through the skin. This can be done by using one light source that has a wavelength within the absorption spectrum of oxygenated blood but not that of non-oxygenated blood, and another light source that has a wavelength within the absorption spectrum of non-oxygenated blood but not that of oxygenated blood. The wavelengths may be, although not necessarily, similar to those used in pulse oximetry, i.e. 660 nm for deoxygenated haemoglobin and 940 nm for oxygenated haemoglobin. The ratio of absorption of the different wavelengths can be used to estimate the amount of heat dissipated versus the amount of heat retained.

Possibly, the afore-described embodiments also allow cardio-activity such as the R to R intervals (RRI) of the pulse to be monitored. From the RRI, the user's heart rate variation (HRV) may be observed over time. HRV refers to the variation in the time intervals between consecutive heartbeats in milliseconds. HRV can be used to indicate the user's state of fatigue, and may provide clues to his state of well-being.

Typically, a small HRV may indicate that the user is feeling uncomfortable or stressed. Conversely, a large HRV may indicate that the user is feeling relaxed or well. If the temperature of a user observed by the embodiment seems to be always higher than the expected 36.9 degrees Celsius (that is, if the embodiment is calibrated to provide this level of accuracy) but the HRV of the user shows that the user is actually feeling relaxed, then this could indicate that the higher temperature is actually normal for the user. Henceforth, the user's future temperature measurements may be referenced to this higher normal temperature. In this way, the embodiment allows temperature monitoring of the user to be personalised based on the user's unique normal temperature.

FIG. 22c is a flowchart that shows an improved method over that e method shown in the flowchart of FIG. 22b, and shows a further step of raising a fourth type of alarm based on monitoring the user's HRV. The fourth type of alarm is raised, at step 2215, if it is decided the user's HRV has decreased, at step 2217. Conversely, the third type of alarm, at step 2213, is raised if it is decided that the user's HRV has not decreased, at step 2217.

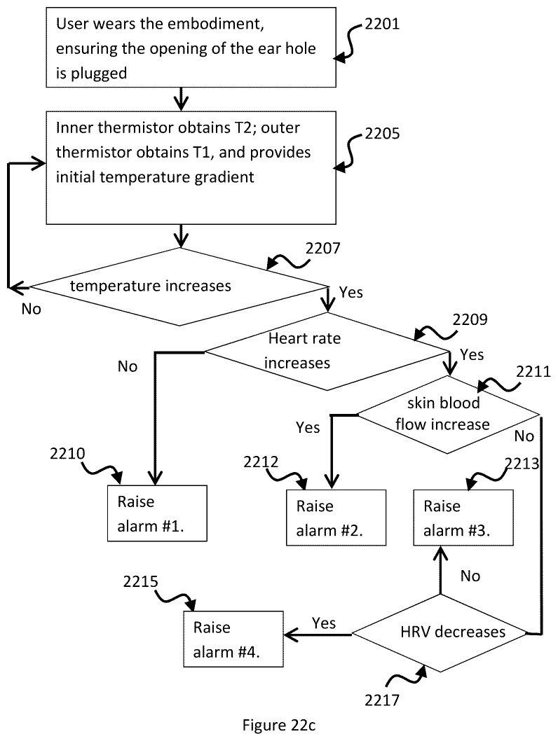

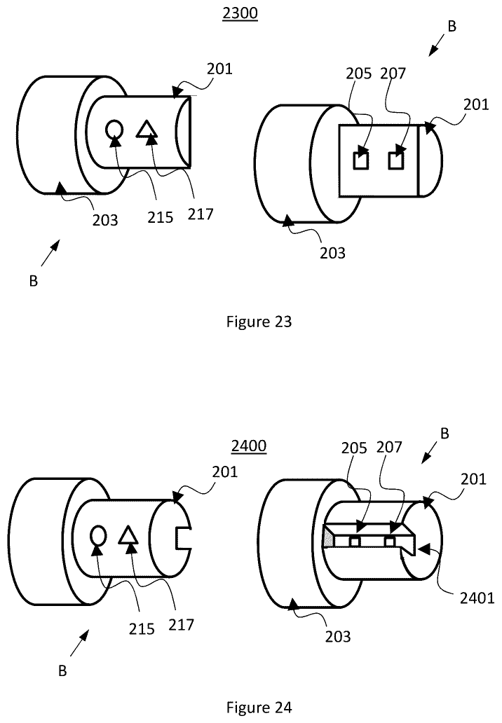

FIG. 23 illustrates a device 2300 which is a variation of the embodiment of FIG. 19, in which one side along the cylindrical extension 201 is hived off, leaving behind an extension 201 having a semi-circular cross-section. The two drawings in FIG. 23 shows two different sides of the same embodiment, the same direction in each drawing indicated is by the arrows marked B. The thermistors 205, 207 are provided on the flat surface of the semi-circular extension 201, which is the side that is nearer the axis of the ear canal and less likely to touch the ear canal wall. Hence, on plugging the device 2300 into the ear of the user, the thermistors 205, 207 are unlikely to be in contact with the ear canal wall, and therefore able to measure temperature of air in the ear canal. The light emitter 215 and optical sensor 217 are placed on the curved side of the extension 201, to increase the likelihood that the light emitter 215 shines directly into the ear canal tissue and that the optical sensor 217 detects mainly light emerging from ear tissue. Preferably, the curved side of the extension 201 is made of a resilient and deformable material that may deform slightly to adapt to the surface relief of the ear canal wall. Foam of most types, such as polyurethane, might be suitable material.

FIG. 24 illustrates yet another device 2400 which is a variation of the embodiment of FIG. 19. This device 2400 has a cylindrical extension 201 that has a groove 2401 cut along the length of the extension 201. The thermistors 205, 207 are placed in the groove 2401 to prevent the thermistors 205, 207 from touching the wall of the ear canal and to measure the temperature gradient along the ear canal. The light emitter 215 and optical sensor 217 are placed outside the groove 2401, on the circumference of the extension 201, in order to be urged against the wall of the ear canal. In this embodiment, the extension does not need to be placed eccentrically to the ear canal axis and also does not need to have a diameter much smaller than the diameter of the ear canal.

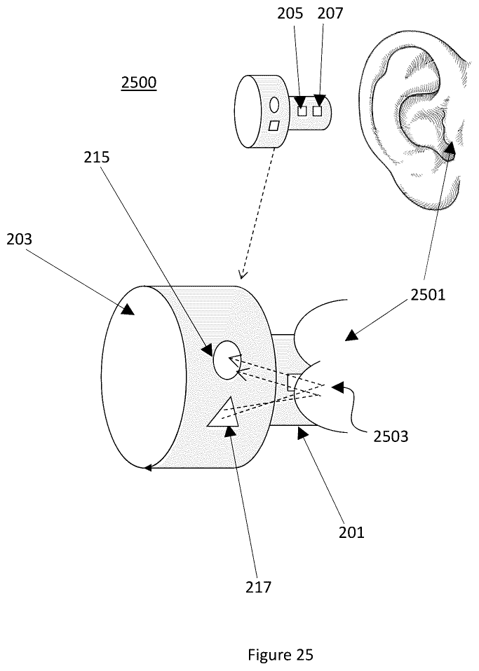

FIG. 25 illustrates yet another device 2500, in which the light emitter 215 and optical sensor 217 are placed on the edge of the plug 203 of the device 2500, and the plug 205 has dimensions that urge the light emitter 215 and optical sensor 217 against the tragus 2501. The tragus 2501 is a small pointed eminence of the external ear, illustrated by an anatomical drawing of the ear in FIG. 25. The light emitter 215 is able to emit light into the tragus 2501 and the optical sensor 217 able to detect light rebounding from within the tragus 2501. The thermistors 205, 207 are arranged on the extension 201 to be inserted into the ear canal and to read the temperature gradient in the ear canal. In this embodiment, the extension is preferably placed eccentrically to the ear canal axis and has a diameter smaller than the diameter of the ear canal.

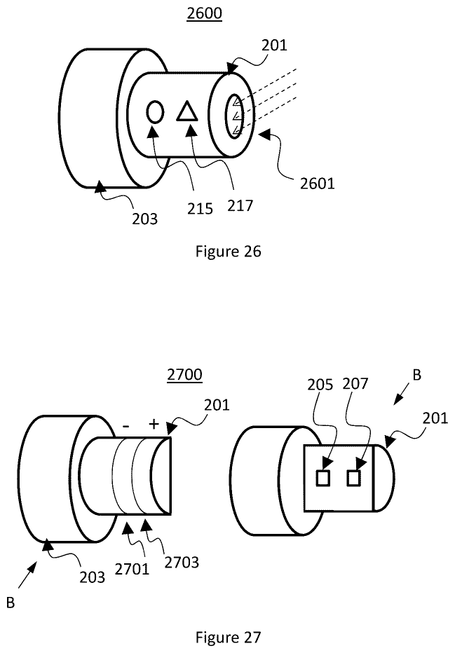

FIG. 26 illustrates yet another device 2600, in which the thermistors 205, 207 are replaced by an infrared temperature monitor 2601. The infrared temperature monitor 2601 is placed at the end of the extension 201 to be pointed towards the inner ear when the device 2600 is worn by the user. This is to detect the infrared radiation emanating from the artery behind the ear drum. However, this device 2600 is not a preferred embodiment as the infrared temperature monitor 2601 leaves no room at the end of the extension for a speaker to be placed, and the device cannot be an earphone. The side of the extension 201 is provided with the light emitter 215 and optical sensor 217. Again, the extension is preferably placed eccentrically to the ear canal axis and has a diameter smaller than the diameter of the ear canal. This embodiment is an example of how body heat monitoring is not confined to use of thermistors to measure temperature gradient in the ear canal.

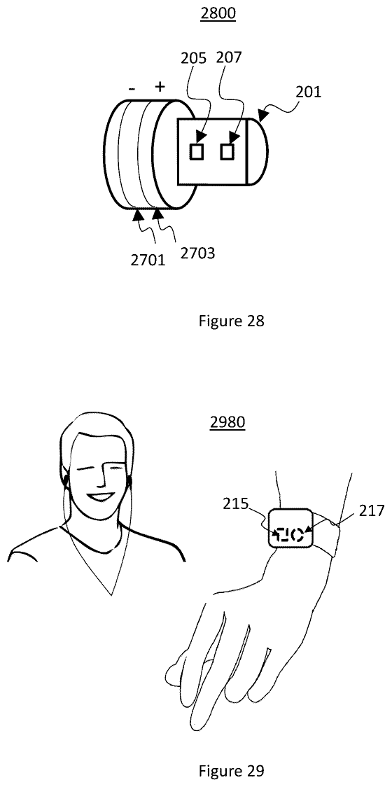

FIG. 27 illustrates yet another device 2700, in which the light emitter 215 and optical sensor 217 are replaced by two lines of electrodes 2701, 2703, one being positive and the other negative, to allow electrocardiographic monitoring of the user's pulse. The embodiment in FIG. 27 is similar to that in FIG. 23, in that one side along the cylindrical extension 201 is hived off, leaving behind an extension 201 having a semi-circular cross-section. The thermistors 205, 207 are also provided on the flat surface of the semi-circular extension 201. The electrodes are provided as two conductive lines 2701, 2703 running circumferentially over the curved part of the extension 201. This embodiment is an example of how heart rate monitoring is not confined to the use of PPG technology. In a variation of this embodiment, as shown in FIG. 28, the two lines of electrodes 2701, 2703 are placed on the edge of the plug 203 to be urged or pressed against the tragus (tragus not illustrated in FIG. 28) to read heart rate.

FIG. 29 illustrates another possible configuration of the embodiment, which is a system 2900 in which the light emitter 215 and optical sensor 217 are worn on the wrist of the user to detect heart rate, while the thermistors (not shown) are arranged on earphones to be placed into the ear canal. This embodiment shows that that the heart rate monitor, i.e. the PPG sensor (photoplethysmography) or electrocardiogram sensor and so on, need not be part of any ear device.

In yet further variations of the embodiments, the heart rate monitor is provided in one ear device, while another ear device comprising a heat monitor such as the thermistors or infrared thermometer. These ear devices operate as a pair and are each worn on the respective ear of the user, and may be connected wirelessly or by a cable.

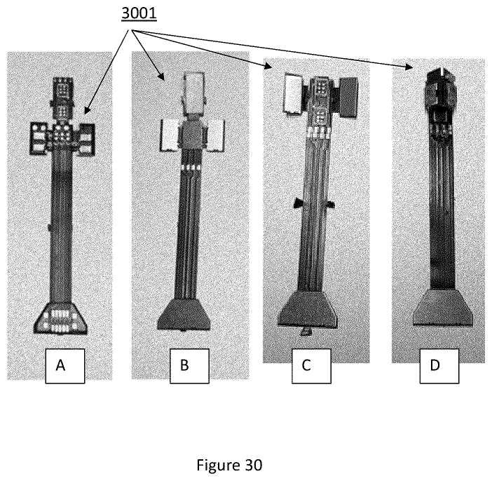

FIG. 30 to FIG. 35 illustrate another embodiment which is a wireless earphone that contains a temperature gradient sensor and a cardiovascular activity monitor. The electrical circuitry in the earphone is partly made up of a flexible printed circuit board (FPC), also commonly called flex circuits. An FPC may be folded into any suitable shape for installation in a device of irregular shape. FIG. 30 illustrates how the FPC 3001 used in the embodiment is folded.

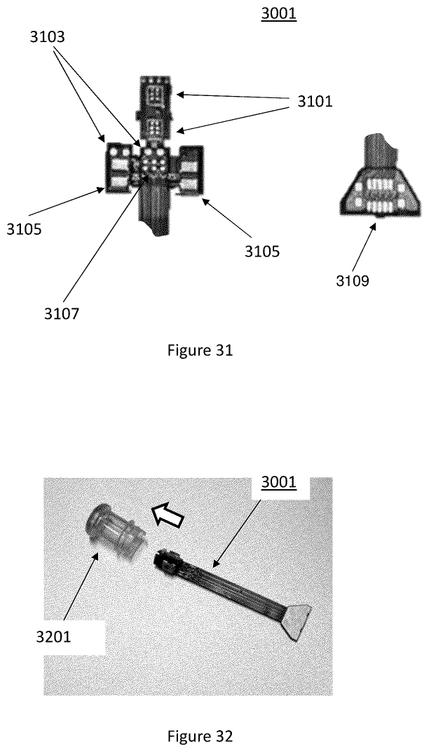

The FPC 3001 shown in FIG. 30 has the basic shape of a cross. It is made of a piece flexible substrate made of a polymeric material such as polyimide, PEEK (polyether ether ketone) or a film of transparent conductive polyester. The substrate is threaded with a conductive paste onto which electronic devices may be printed, typically by stencilling. The threads provide the circuit connections or lines typically seen in printed circuit boards. There are four pictures in FIG. 30, arranged into picture A on the leftmost and progressing to picture D on the rightmost, each progression showing a step in the configuring of the FPC 3001. FIG. 31 comprises enlarged images of the head of the FPC 3001 and the foot of the FPC 3001.

FIG. 30A shows the underside of the FPC 3001, which is installed with two semiconductor temperature sensors 3101 for detecting temperature gradient in the air of the user's ear canal.

PPG sensors are also provided on the FPC 3001. The PPG sensor is made up of two IR LEDs (infrared light emitting diode) and two corresponding photodiodes. On one arm of the cross and on the neck of the head are placed an IR LED 3103 each. On each arm is placed a photodiode 3105. The preferred photodiode is the NJL6193R-3 model of the brand JRC Electronics Devices. The heart of the cross comprises miscellaneous components such as resistors, capacitors and any other sensors. The foot 3109 of the cross is a connecter for connecting the FPC 3001 to another printed circuit board that is also within the earphone. The foot 3109 may comprises a microprocessor to control the components on the FPC 3001.

In some other embodiments, light emitting diodes of other emission wavelengths may be added to the FPC 3001 for measurement of other types of physiological data, such as wavelengths suitable for measuring blood for characteristics of oxygen saturation or blood pressure.

FIG. 30B shows the topside of the FPC 3001. The topside of each arm of the cross is applied with a tiny piece of metal plates to provide rigidity. The topside of the head of the cross is applied with an adhesive, such as double-sided adhesive tape.

FIG. 30C shows the topside of the head folded back and stuck onto the topside of the heart by the adhesive tape, such that the two semiconductor temperature sensors 3101 face away from the heart. The two arms are bent to lean towards each other and over the heart. The bent arms define a space above the semiconductor temperature sensors 3101, which provides a protected, in-situ air space for monitoring the temperature gradient in the ear canal.

Accordingly, this shows how rigidized portions in an FPC can be folded to provide a shell, into which electronic components on the FPC that are sensitive to contact may be placed for protection, such as optical sensors, temperature sensors, piezoelectric components, pyroelectric components and so on. Moreover, providing space around sensitive components allows better drying of damp that might have crept into the component.

Hence, the shell made by folding a FPC with rigidized portions offers protection that improves the efficiency and lifespan of such touch sensitive components.

FIG. 32 shows how the FPC 3001 with arms and head folded is able to fit into an optical head 3201 of a wireless earphone. The optical head 3201 is typically a plastic cap which allows light from the light source to shine through to reach the ear canal wall. The two semiconductor temperature sensors are arranged in the optical head 3201 in such a way that one would be placed deeper into the ear canal than the other when the wireless earphone is worn by the user.

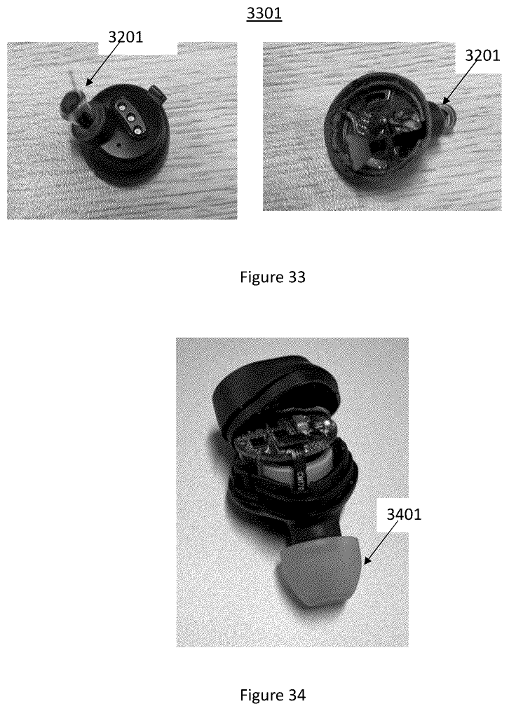

FIG. 33 shows the front casing of the wireless earphone, into which the optical head has been inserted. The picture on the left shows the frontal view of the front casing, while the picture on the right shows the underside view of the front casing.

FIG. 34 shows the assembled wireless earphone opened to reveal the electronic components inside. The front casing shown in FIG. 33 is not easily visible in FIG. 34. However, the skilled reader would appreciate that the optical head 3201 is inside the earbud 3401 of the wireless earphone as shown.

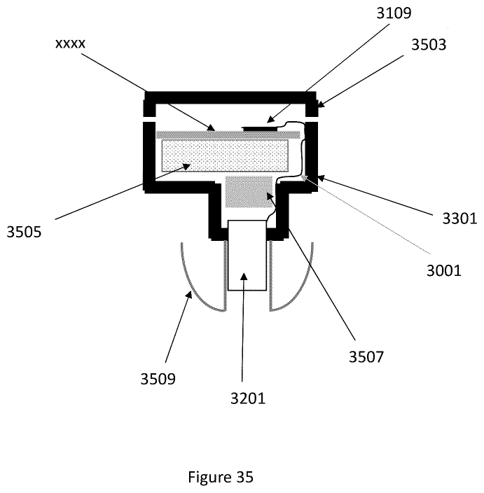

FIG. 35 is a schematic illustration of the lateral cross-section of the earphone shown in FIG. 34. It is clearly visible in FIG. 35 that the casing comprises of two parts, a front casing 3301 (also shown in FIG. 33) and a back casing 3503. The front casing 3301 and back casing 3503 can be joined to form an assembled casing.

The FPC 3001 is folded in order that it may be tucked inside the assembled casing. The connector at the foot 3109 of the FPC 3001 is connected to a main printed circuit board (PCB) in the wireless earphone. The PCB typically comprises a microprocessor, a wireless transceiver for data transfer with a playback device such as a smartphone and other wireless earphone components as may be required. The assembled wireless earphone comprises a battery 3505. Between the battery and the optical housing is an acoustic speaker 3507. The assembled casing has an opening through which the optical housing 3201 is inserted. Around the optical housing 3201 is a layer of soft elastic ear gel 3509, typically of silicone. The ear gel is transparent to the wavelength of light emitted by the IR LEDs 3103. Unlike the earlier mentioned embodiments, the light emitters (i.e. the IR LEDs 3103) and the optical sensors (i.e. the photodiode 3105) are not placed in direct physical contact with the ear canal wall. Nevertheless, the ear gel 3509 around the optical housing 3201 allows good transmission of light from the IR LEDs 3103 to the ear canal wall with low rate of scattering within the ear gel 3509. Hence, most of the light enters the canal wall on passing through the ear gel, and on rebounded from within the ear tissue, some of the light pass through the ear gel to be detected by the photodiode 3105.

Furthermore, the skilled reader would appreciate that embodiments which measure the temperature gradient of the ear canal wall, i.e. by physically touching the ear canal wall with thermistors, are within the contemplation of this application. That is, one thermistor touches the ear canal wall nearer the mouth of the canal while other touches the wall in a deeper part of the ear canal. However, it is difficult to design an ear device that fits the many different shapes and sizes of ear holes such that the thermistors are consistently and firmly pressed against the ear canal wall. In contrast, measuring temperature gradient of the air in ear canal does not have this problem.

Therefore, the embodiments include an earplug 1900 comprising a heat detector (205, 207, 2601); a pulse monitor (i.e. a heart rate sensor 215, 217, 2701, 2703); wherein the heat detector is capable of obtaining an indication of the level of heat in the air of the ear canal of a subject, i.e. the user, wearing the earplug at the same time as the pulse monitor is obtaining an indication of the heart rate of the subject.

Also the embodiments include a method for monitoring a subject of his body heat, comprising the steps of: obtaining an indication of the level of heat in the ear canal of the subject at the same time as obtaining the heart rate of the subject; and raising an alert if the heart rate is higher than a pre-determined upper threshold heart rate for the level of heat in the ear canal; or raising an alert if the heart rate is lower than a pre-determined lower threshold heart rate for the level of heat in the ear canal.

The embodiments also include a method for monitoring insufficient body heat dissipation in a subject, comprising the step of: raising an alert if an increase in temperature of the subject is not accompanied by a concurrent and sufficient increase in subcutaneous blood flow to a level pre-determined for the extent of the increase in temperature.

Furthermore, the embodiment include a a folded printed circuit board 3001 comprising: a substrate imprinted conductive lines to provide electrical circuitry; one or more portions of the substrate being relatively flexible portions; and one or more portions of the substrate being relatively rigid portions; wherein the substrate folded such that the relatively rigid portions define a space for accommodating electronic components on the substrate.

The embodiments also include an earbud 3401 comprising: a bud for placement into an ear canal when the earbud is worn by a subject; the folded printed circuit board having a space for accommodating electronic component is inside the bud.

While there has been described in the foregoing description preferred embodiments of the present invention, it will be understood by those skilled in the technology concerned that many variations or modifications in details of design, construction or operation may be made without departing from the scope of the present invention as claimed.

For example, although light transmission through ear tissue has been described in the main, the skilled reader would understand that light reflectance by ear tissue is within the contemplation of this description in variations of the embodiments described.

Furthermore, instead of photoplethysmography, ballistocardiography may be used. As the skilled reader would know, ballistocardiography is a technique for producing a graphical representation (displacement, velocity or acceleration) of minute, involuntary repetitive motions of the human body arising from the pulsation of blood into the great vessels. In other words, it is an integration of multiple forces related to movements of blood inside the heart, inside the arteries and movement of the heart itself. It is can be a three dimensional signal, although most measurement techniques simply measure the longitudinal, head-to-toe component.

Also, the adhesive tape used in the FCP of FIG. 30 can be replaced by a layer of pressure sensitive adhesive. Alternatively, the bend of the head can be provided and secured by partially making the cross out of a less resilient but malleable material. Any option for securing the head bending over, even any mechanical structure for the purposes such as a miniature clasps, is within contemplation.

In other embodiments, the FPC shown in FIG. 30, along with electronic components installed on to the FPC, may be wrapped around a Balanced Armature Receiver. A Balanced Armature Receiver is typically shaped as a rectangular prism. In this case, the FPC consists of four folds instead of three folds with one light source or one photodiode placed on one of the folds.

Embodiments in which subcutaneous blood content or blood flow of the user may be used independently and separately to monitor the heat condition of the user, i.e. effectiveness in heat dissipation only, are within the contemplation of the embodiments.

Also, embodiments in which subcutaneous blood content or blood flow of the user may be monitored in tandem with the user's pulse only are within the contemplation of the embodiments.

Also, embodiments in which subcutaneous blood content or blood flow of the user may be monitored in tandem with the user's temperature only are within the contemplation of the embodiments.

Also, embodiments in which subcutaneous blood content or blood flow of the subject may be monitored in tandem with both the subject's pulse and the subject's temperature, are all within the contemplation of this application.

1. A earplug comprising
a heat detector;
a pulse monitor; wherein
the heat detector is capable of obtaining an indication of the level of heat in the air of the ear canal of a subject wearing the earplug at the same time as the pulse monitor is obtaining an indication of the heart rate of the subject.

2. A earplug as claimed in claim 1, wherein
the heat detector is formed of at least two heat sensors arranged to be spaced apart by a pre-determined distance such that
the at least two heat sensors are capable of measuring the temperature of the air in at least two respective locations in the ear canal; wherein
the temperature of the air in at least two respective locations being suitable for deducing the temperature gradient in the ear canal to provide the indication of the level of heat in the ear canal.

3. A earplug as claimed in claim 2, wherein
the at least two heat sensors are semiconductor temperature sensors.

4. A earplug as claimed in claim 1, wherein
the pulse monitor is a photoplethysmography sensor or a ballistocardiography sensor.

5. A earplug as claimed in claim 1, comprising:
an extension suitable for placement into the ear canal;
the extension having a first side and a second side;
the first side and the second side having dimensions such that the first side is closer to the ear canal wall and the second side is further from the canal wall;
the pulse monitor being placed on the first side; and
the heat detector being placed on the second side.

6. A earplug as claimed in claim 5, comprising
the second side is in a depression on the surface of the extension.

7. A earplug as claimed in claim 1, comprising:
an extension for placement into the ear canal, the heat detector being on the extension;
earplug having a plug portion for remaining outside the ear canal when the earplug is worn by the subject, the pulse monitor being on the plug portion;
the plug portion having dimensions suitable for urging the pulse monitor into contact with the tragus of the ear when the extension is placed into the ear canal; wherein
the indication of the heart rate of the subject is obtained from the tragus of the ear of the subject.

8. A earplug as claimed in claim 1, comprising:
a flexible printed circuit board;
the heat detector and the pulse monitor being components on the flexible printed circuit board;
the flexible printed circuit board folded to define a space for accommodating the heat detector, and the flexible printed circuit board folded to fit inside the earplug; such that
when the earplug is worn by the subject, the heat detector is placed into the ear canal.

9. A earplug as claimed in claim 8, wherein
the flexible printed circuit board is reinforced by one or more layers of a hard substrate such that a respective one or more portions of the flexible printed circuit board are more rigid than the other portions of the flexible printed circuit board; wherein
the flexible printed circuit board is folded such that the rigid portions of printed circuit board cooperate to define the space for accommodating the heat detector.

10. A method for monitoring a subject of his body heat, comprising the steps of:
obtaining an indication of the level of heat in the ear canal of the subject at the same time as obtaining the heart rate of the subject; and
raising an alert if the heart rate is higher than a pre-determined upper threshold heart rate for the level of heat in the ear canal; or
raising an alert if the heart rate is lower than a pre-determined lower threshold heart rate for the level of heat in the ear canal.

11. A method for monitoring a subject of his body heat as claimed in claim 10, wherein
the heart rate is obtained by a photoplethysmography sensor.

12. A method for monitoring a subject of his body heat as claimed in claim 10, wherein
the indication of the level of heat is the temperature gradient in the canal of the ear.

13. A method for monitoring a subject of his body heat as claimed in claim 12, wherein obtaining an indication of the level of heat in the ear canal further comprises the steps of:
detecting a change in the temperature gradient in the canal of the ear.

14. A method for monitoring a subject of his body heat as claimed in claim 10, further comprising the steps of:
observing the indication of the level of heat at the same time as observing the amount of subcutaneous blood in the subject; and
raising an alert if the heart rate is higher than a pre-determined upper threshold heart rate for the level of heat in the ear canal without an accompanying increase in the amount of subcutaneous blood in the subject to a pre-determined threshold amount.

15. A method for monitoring a subject of his body heat as claimed in claim 14, wherein the step of observing the amount of subcutaneous blood of the subject over the same period of time comprises:
observing the amount of absorption of a light transmitted into the skin of the subject.

16. A method for monitoring a subject of his body heat as claimed in claim 15, wherein the step of observing the amount of subcutaneous blood of the subject comprises:
observing the ratio of deoxygenated haemoglobin to oxygenated haemoglobin;
taking an increase in the ratio of deoxygenated haemoglobin to oxygenated haemoglobin to mean an increase in subcutaneous blood; and
taking a decrease in the ratio of deoxygenated haemoglobin to oxygenated haemoglobin to mean a decrease in subcutaneous blood.

17. A method for monitoring insufficient body heat dissipation in a subject, comprising the step of:
raising an alert if an increase in temperature of the subject is not accompanied by a concurrent and sufficient increase in subcutaneous blood flow to a level pre-determined for the extent of the increase in temperature.

18. A method for monitoring body heat dissipation in a subject as claimed in claim 17, further comprising the step of:
observing heart rate of the subject; and
requiring a concurrent increase in heart rate before the alert is raised.

19. A method for monitoring body heat dissipation in a subject as claimed in claim 17, further comprising the step of:
observing heart rate variation of the subject; and
requiring a concurrent reduction in the heart rate variation before the alert is raised.

20. A method for monitoring a subject of his body heat, comprising the steps of:
obtaining an indication of the level of heat in the ear canal of the subject at the same time as obtaining the heart rate of the subject; and
raising an alert if the heart rate is lower than a pre-determined upper threshold heart rate variation (HRV) for the level of heat in the ear canal.

21. A folded printed circuit board comprising:
a substrate imprinted conductive lines to provide electrical circuitry;
one or more portions of the substrate being relatively flexible portions; and
one or more portions of the substrate being relatively rigid portions; wherein
the substrate folded such that the relatively rigid portions define a space for accommodating electronic components on the substrate.

22. A folded printed circuit board as claimed in claim 21, wherein
the rigidity of the remainder of the substrate is provided by application of a layer of rigid material to the substrate.

23. A folded printed circuit board as claimed in claim 22, wherein
the layer of rigid material is metal.

24. A folded printed circuit board as claimed in claim 20, wherein
electronic components include at least two heat detectors.

25. A folded printed circuit board as claimed in claim 24, wherein
the at least two heat detectors are semiconductor temperature sensors.

26. A earbud comprising:
a bud for placement into an ear canal when the earbud is worn by a subject;
the folded printed circuit board as claimed in claim 20; wherein
the space for accommodating electronic component is inside the bud.

27.-31. (canceled)
